(19) United States
(12) Patent Application Publication
(10) Pub. No.: US 2018/0325700 A1
(43) Pub. Date: Nov. 15, 2018

(54) BREAST PROSTHESIS

(71) Applicant: Amoena Medizin-Orthopädie-Technik GmbH, Raubling (DE)

(72) Inventor: Nils SELTER, Frasdorf (DE)

(21) Appl. No.: 15/775,191

(22) PCT Filed: Nov. 11, 2016
(86) PCT No.: PCT/EP2016/001880

§ 371 Date: May 10, 2018

(30) Foreign Application Priority Data
Nov. 13, 2015 (DE) 202015007895.8

(51) Int. Cl. A61F 2/52; A61F 2/12

(52) U.S. Cl. CPC A61F 2/52; A61F 2002/523; A61F 2/12

(57) ABSTRACT

The invention relates to a breast prosthesis having an elastically deformable outer body whose outer surface is patterned on the shape of the breast and having a plastically deformable inner body whose inner surface can adapt to irregularities of the tissue. In accordance with the invention, the inner surface of the inner body has a three-dimensional structure and/or is provided with a textile that is designed such that a ventilation of the tissue surface can be achieved.

The invention relates to a breast prosthesis having an elastically deformable body whose outer surface is patterned on the shape of the breast.

Breast prostheses are used for cosmetic reasons after (partial) removals of breast typically due to cancer. A high comfort in wear should in particular be achieved in addition to a shape and feel that comes as close as possible to the natural breast.

It is known from the prior art to provide the inner side of the prosthesis with a plastically deformable layer that adapts to the shape of the scar tissue. Documents EP 0 768 068 A1, EP 2 133 042 A1 or WO 2014/078021 A1 can be named in this connection, for example.

With these known prostheses, however, the problem can occur that the scar tissue was occlusively closed by this plastically deformable layer. This can result in heat accumulations and the formation of perspiration beneath the prosthesis, which is detrimental to the comfort in wear and can also result in skin reactions.

It is the object of the invention to provide a breast prosthesis whose inner side can adapt to the shape of the scar tissue without preventing an exchange of air.

Against this background, the invention relates to a breast prosthesis having an elastically deformable outer body whose outer surface is patterned on the shape of the breast and having a plastically deformable inner body whose inner surface can adapt to irregularities of the tissue. Provision is made in accordance with the invention that the inner surface of the inner body has a three-dimensional structure and/or is provided with a textile that is designed such that a ventilation of the tissue surface can be achieved.

In an embodiment, the outer body and the inner body contact one another directly.

In an embodiment, the breast prosthesis consists of the outer body, the inner body, and the structure or the textile.

In an embodiment, the outer body is of shell shape.

In an embodiment, the outer body has a rubber-elastic compound enclosed in an outer chamber. The outer chamber can, for example, be formed by a film bag. The rubber-elastic compound can, for example, be a two-component silicone rubber compound.

In an embodiment, the inner body has a plastically deformable compound enclosed in an inner chamber. The inner chamber can, for example, also be formed by a film bag.

The plastically deformable compound can be a homogeneous or heterogeneous compound. The compound can have thixotropic properties. The compound can be a shear thinning compound having a yield point. Examples include low-cured silicone gels, preferably those having at least two components that can optionally further contain one or more additives. Suitable additives include phase change materials, expanded lightweight fillers, for example porous or gas-filled particles (non-expanded lightweight fillers can be added as part of the manufacturing process that expand due to temperature during the manufacturing process), thickening agents, aerosols or thixotropic agents. Examples of further suitable plastically deformable compounds are disclosed in EP 0 768 068 A1, EP 2 133 042 A1 and WO 2014/078021 A1.

In an embodiment, the film bags are formed by two plastic films welded along the common periphery. Provision can be made that the inner boundary film of the outer body corresponds to the outer boundary film of the inner body and that the outer and inner chambers are formed by three plastic films welded along the common periphery.

The three-dimensional structure or the textile is preferably plastically and/or elastically deformable. It is shaped such that a ventilation of the skin is ensured.

In an embodiment, the three-dimensional structure is molded to the inner surface of the inner body. In another embodiment, the three-dimensional structure is worked into the inner surface of the inner body. Provision can, for example, be made that the three-dimensional structure forms an integral part of the plastic film that represents the inner boundary of the inner body. It can be molded to this film or can be worked into it.

In an embodiment, the three-dimensional structure is adhesively bonded or welded to the inner surface of the inner body.

In an embodiment, the three-dimensional structure is a structure composed of plastic. The plastic material can be the same material that is used for the plastic film that represents the inner boundary of the inner body. Alternatively, different plastic materials can be used for the film and for the structure.

In an embodiment, the three-dimensional structure comprises channels, webs and/or nubs.

In an embodiment, the textile is adhesively bonded or welded to the inner surface of the inner body.

In another embodiment, the textile is attached to the inner surface of the inner body using a holding means. Provision can, for example, be made that the textile is attached to the inner surface of the inner body using a hook and loop surface adhesively bonded or welded to the inner surface of the inner body.

In an embodiment, the textile is a three-dimensional textile having a thickness of, for example, greater than 2 mm and preferably of greater than 5 mm. Examples include two-surface or multi-surface textiles such as spacer fabrics or knitted spacer fabrics.

In an embodiment, the textile has a surface structure such as ribs or nubs at at least the surface facing the body.

In an embodiment, the textile comprises or consists of natural fibers. Natural fibers can have advantages in skin compatibility and in low odor take-up. Examples of suitable natural fibers include cotton fibers or wool fibers.

In an embodiment, the textile comprises or consists of plastic fibers. Plastic fibers can have advantages in good perspiration removal and low surface roughness.

Combinations of natural fibers and plastic fibers are also conceivable and covered by the invention.

Further details and advantages result from the embodiments discussed in the following with reference to the Figures. There are shown in the Figures:

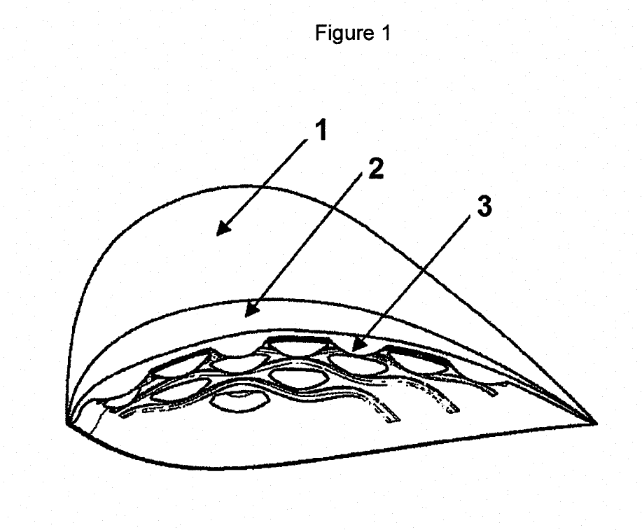

FIG. 1: a perspective view of a breast prosthesis in accordance with the invention;

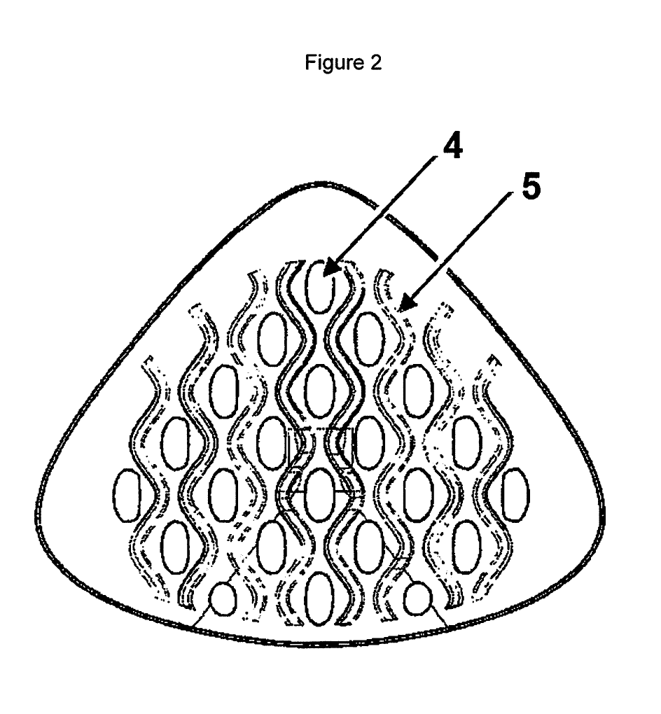

FIG. 2: a plan view of the inner surface of the breast prosthesis shown in

FIG. 1; and

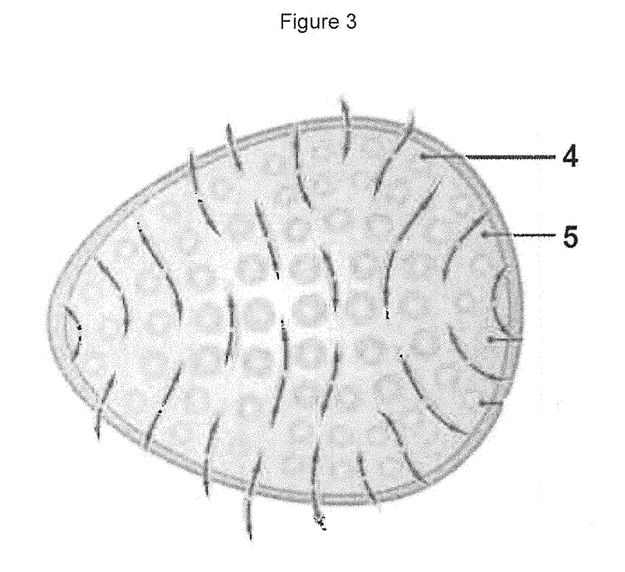

FIG. 3: a plan view of the inner surface of a further embodiment of a breast prosthesis in accordance with the invention.

FIG. 1 shows a perspective view of a breast prosthesis in accordance with the invention. The outer side of the breast prosthesis is formed by a shell-shaped outer body 1 that is elastically deformable. A likewise shell-shaped inner body 2 is attached to the inner side of this shell-shaped outer body 1.

The outer body 1 comprises two plastic films that are welded along the peripheral margin and that form a chamber in which a rubber-elastic two-component silicone rubber compound is located.

The inner body 2 is formed by welding a further film to the inner side of the outer body 1 and by filling the interposed chamber with a filler material. The filler material is a plastically deformable compound having thixotropic material properties which can be a low-cured silicone gel of two different silicone components that further contains phase change material, expanded lightweight fillers, thickening agents, aerosols, and thixotropic agents.

A structured plastic inlay 3 is adhesively bonded to the inner film of the plastically deformable body 2. The adhesive surface of the inlay 3 is smooth to ensure good adhesion to the body 2.

The surface of the inlay 3 facing the body has a three-dimensional structure whose design can be seen from FIG. 2. The structure comprises a regular arrangement of nubs 4 and ventilation channels or folding channels 5. The nubs 4 are arranged in a plurality of rows extending in parallel, with the nubs being offset by half a nub spacing in adjacent rows. All the nubs 5 are of identical design and have a design rounded in hill shape and having an oval base surface. The channels 5 extend in zigzag shape between the individual nub rows.

A further embodiment of a breast prosthesis in accordance with the invention is shown in FIG. 3 whose three-dimensional surface structure differs from that of the embodiment in accordance with FIGS. 1 and 2.

In the embodiment in accordance with FIG. 3, the nubs 5 are sectionally arranged in curved rows and sectionally evenly distributed. They have a design rounded in hill shape and having a circular base surface. The size of the nubs 5 decreases from the center of the support surface toward the margin of the support surface. Slits 4 of a defined length are worked into the inlay 3 between the nubs 5 and serve the ventilation of the tissue and the better deformability of the inlay 3. Flow paths of the air through the structure are indicated by arrows in FIG. 3.

An adaptation to the irregular scar tissue can take place by the provision of both a plastically deformable layer 2 and a three-dimensionally shaped ventilation layer 3 without the air supply to the tissue being interrupted.

1. A breast prosthesis for use with a breast having a shape, the breast prosthesis comprising
1. an elastically deformable outer body, the outer body having an outer surface, the outer surface being patterned on the shape of the breast and
2. a plastically deformable inner body having an inner surface adaptable to irregularities of underlying tissue,
wherein the inner surface of the inner body has a three-dimensional structure and/or is provided with a textile that is designed such that a ventilation of the tissue surface can be achieved.

2. A breast prosthesis in accordance with claim 1, characterized in that the outer body is of shell shape.

3. A breast prosthesis in accordance with claim 1, wherein the inner body has an inner chamber containing a plastically deformable compound.

4. A breast prosthesis in accordance with claim 1, wherein the three-dimensional structure is molded to the inner surface of the inner body or is worked into it.

5. A breast prosthesis in accordance with claim 1, wherein the three-dimensional structure is adhesively bonded or welded to the inner surface of the inner body.

6. A breast prosthesis in accordance with claim 1, wherein the three-dimensional structure is a structure composed of plastic.

7. A breast prosthesis in accordance with claim 1, wherein the three-dimensional structure comprises channels, webs and/or nubs as structural elements.

8. A breast prosthesis in accordance with claim 1, wherein the textile is adhesively bonded or welded to the inner surface of the inner body.

9. A breast prosthesis in accordance with claim 1, wherein one the textile is a three-dimensional textile having a thickness of greater than 2 mm.

10. A breast prosthesis in accordance with claim 1, wherein the textile comprises natural fibers or plastic fibers.

11. A breast prosthesis in accordance with claim 1, wherein the outer body has an outer chamber containing a rubber-elastic compound.

12. A breast prosthesis in accordance with claim 11, wherein the outer chamber is formed by a film bag.

13. A breast prosthesis in accordance with claim 11, wherein the rubber-elastic compound is a two-component silicone rubber compound.

14. A breast prosthesis in accordance with claim 3, wherein the inner chamber is formed by a film bag.

15. A breast prosthesis in accordance with claim 8, wherein the textile is attached to the inner surface of the inner body using a holding means.

16. A breast prosthesis in accordance with claim 8, wherein the textile is attached to the inner surface of the inner body by a hook and loop surface adhesively bonded or welded to the inner surface of the inner body.

17. A breast prosthesis in accordance with claim 8, wherein the textile is attached to the inner surface of the inner body.

18. A breast prosthesis in accordance with claim 9, wherein the textile has a thickness of greater than 5 mm.

19. A breast prosthesis in accordance with claim 9, wherein the textile is a two-surface textile which is a spacer fabric that is optionally knitted and optionally has a surface structure comprising ribs or nubs.

20. A breast prosthesis in accordance with claim 19, wherein the textile is a multi-surface textile.
